(19) United States
(12) Patent Application Publication
(10) Pub. No.: US 2005/0080354 A1
(43) Pub. Date: Apr. 14, 2005

(54) Ear thermometer with illumination

(76) Inventor: David W. Crossley, Charlestown, RI (US)

Correspondence Address: James S. Pristelski;Cook, Alex, McFarron, Manzo, Cummings & Mehler, Suite 2850, 200 West Adamds, Chicago, IL, 60606, US

(21) Appl. No.: 10/683,320

(22) Filed: Oct. 10, 2003

(51) Int. Cl. A61B 5/00

(52) U.S. Cl. 600/549

(57) ABSTRACT

An ear thermometer has a light source to illuminate the field in front of the tip of the probe, such as the area around the ear canal, or the entire ear, to assist in insertion of the probe into the ear canal. The light source may consist of an incandescent lamp or one or more LEDs of the bright type, and application of the illumination may be controlled by a user activated switch to conserve battery life. The light source may be disposed in a raised protrusion on the housing of the thermometer such as near the top of the housing to supply divergent rays of light to the field of interest, such as the ear.

### FIELD OF THE INVENTION

This invention generally relates to measurement of the body temperature of a human with an ear thermometer. More particularly, this invention relates to an ear thermometer with an illumination feature.

### BACKGROUND OF THE INVENTION

Infrared thermometers for measuring the temperature of the human body are in wide-spread use. Such thermometers are typically inserted partially into the ear canal to measure the temperature in the interior of the ear, and, hence, the body temperature.

Some prior art patents disclose the use of illumination in an ear instrument. U.S. Pat. No. 4,494,881 to Everest discloses a sighting system for an infrared thermometer that provides a visible light beam that is parallel with path of the infrared rays through the optics of this system. The optics in this system are complex and relatively expensive, such as Cassegrainian lens systems, Fresnel lenses, beam splitters and the like. This system also has the undesirable effects of introducing visible light energy into the ear canal, which can interfere with accurately measuring the temperature with infrared techniques. Furthermore, the visible light for guiding the probe of the infrared thermometer into the ear canal must be seen from the side since the body or housing of the thermometer blocks any direct view into the ear canal as the probe nears the ear. Thus, if the light beam of Everest is directly into the ear canal, it may be difficult to see it from the side. Thus, use of a narrow and apparently collimated visible light beam directly into the ear canal, as proposed by Everest, is not an effective means of assisting the insertion of the probe of the thermometer into the ear canal.

U.S. Pat. No. 5,209,757 to Klug et al. teaches an illuminated ear cleaning device with an incandescent light bulb at the end of a tube near a cleaning tip. However, this technique cannot be applied to an infrared thermometer because the light bulb would block the end of the probe through which infrared radiation needs to be received by the thermometer. Moreover, if the radiation emitted by the light bulb is in the path of the infrared radiation from the ear, inaccuracies would be introduced in measuring the infrared radiation.

Many persons, including medical personnel, prefer infrared ear thermometers over electronic oral thermometers because of the convenience and the rapid temperature measurement that the infrared ear thermometers offer. Typically, a hygienic plastic sleeve may be placed over the tip of the probe before each use of the thermometer, the probe itself may be disposable, or the tip of the probe can be cleaned with alcohol.

However, there is often a need to measure temperature with an ear thermometer when the room is dark, such as at nighttime or in rooms with poor ambient light levels. For example, it may be desirable to take the temperature of an infant, child or adult without disturbing their sleep, as by turning on lights in the room. But, without adequate light, it is also difficult to properly position the ear thermometer near the ear and into the ear canal of the person.

There is therefore a need for an ear thermometer with a light feature that provides enough illumination to properly position the thermometer in or near the ear, without flooding the room with light that may be annoying to the person.

It is therefore a general object of the present invention to provide an ear thermometer with an illumination feature to provide sufficient illumination to properly position the ear thermometer for insertion of the probe tip into the ear.

It is another object of the present invention to provide such an illumination feature that is integral with the ear thermometer and easy to use.

A further object of the present invention is to provide such an illumination feature for an ear thermometer that may be selected when desired or needed.

Yet another object of the present invention is to provide an illumination feature wherein the direction of the illumination is forward of the probe tip of the ear thermometer.

Another object of the present invention is to provide a divergent pattern of illumination from a light source for illuminating at least the area of the ear around the ear canal, and, preferably, the entire ear.

### SUMMARY OF THE INVENTION

The present invention is directed to an improved ear thermometer with illumination to assist in inserting a probe of the thermometer into the ear canal of a human being. The thermometer has a housing that may consist of multiple housing portions. A display for displaying a measured temperature is disposed on a rear side of the housing. A tapered probe is disposed on the front side of the housing, typically at an oblique angle thereto. Preferably, the probe is also pivotally and/or rotationally mounted to the front side of the housing to permit some movement of the housing of the thermometer while the probe is inserted into the ear canal. A display is also disposed on the housing for displaying a measured temperature.

In accordance one aspect of the present invention, a light source is disposed on the housing to supply illumination in front of the tip of the probe to illuminate at least the area around the ear canal as the tip of the probe is being inserted therein. A separate switch is preferably disposed on the housing to control when the light source is actuated, thereby conserving battery power. The light source may include an incandescent lamp or one or more light emitting diodes (LEDs) of the bright type. The illumination provided by the light source is in the form of divergent rays to illuminate a substantial area of the ear, and preferably the entire ear.

In accordance with another aspect of the present invention, the light source may be disposed in a raised protrusion on the housing, such as at the top end thereof. The illumination from the light source is generally directed forwardly of the tip of the probe to illuminate the field in front thereof. An axis may be defined through the probe. At least some of the illumination provided by the light source will intersect with the axis in front of the tip of the probe.

### BRIEF DESCRIPTION OF THE DRAWINGS

The features of the present invention which are believed to be novel are set forth with particularity in the appended claims. The invention, together with the further objects and advantages thereof, may best be understood by reference to the following description taken in conjunction with the accompanying drawings, in the several figures in which like reference numerals identify like elements, and in which:

FIG. 1 is a back elevational view of a first embodiment of an ear thermometer in accordance with the present invention;

FIG. 2 is a side elevational view of the first embodiment of the ear thermometer shown in FIG. 1;

FIG. 3 is a front elevational view of the first embodiment of the ear thermometer shown in FIGS. 1 and 2; and

FIG. 4 is an electronic schematic diagram of the circuitry utilized in the electronic thermometers of the present invention.

### DETAILED DESCRIPTION OF THE INVENTION

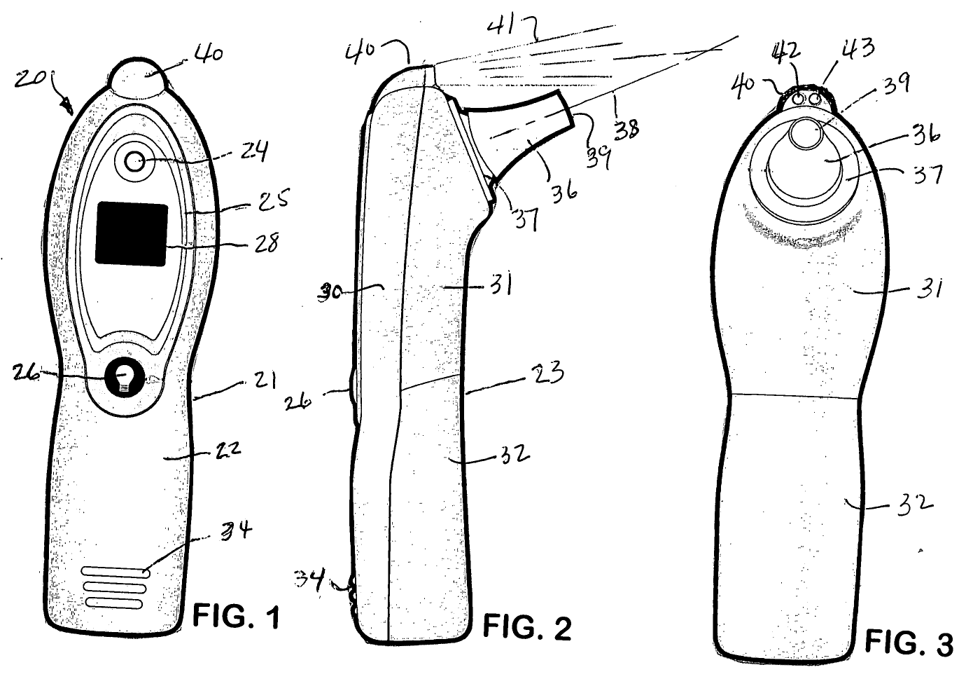

Referring to the Figures, and particularly to FIG. 1, a preferred embodiment of an ear thermometer, generally designated 20, is constructed in accordance with the present invention. The operative controls, including a power on/off switch 24 and an illumination switch 26 are disposed on the backside 22 of the housing 21 of thermometer 20. Switches 24 and 26 may be of the push button type. In the illustrated embodiment of FIG. 1, a display 28 is disposed between switches 24 and 26. However, it will be readily apparent that the location of switches 24 and 26 and display 28 may be arranged in other configurations and/or locations on the housing 21. A decorative bezel 25 may be disposed around some or all of the switches 24 and 26 and display 28, if desired.

Display 28 may of the liquid crystal display (LCD) type and provide a variety of indicia for informing the user of the temperature measurements and of the operating modes. For example, it may have numerical indicia to numerically display a measured temperature and it may also have alpha indicia, such as an “L” to indicate that the measured temperature is below the normal temperature range such as below about 32° C. (89.6° F.), and an “H” to indicate that the measured temperature is above the normal temperature range such as about 43° C. (109.4° F.). Display 28 may also have indicia, such as “C” to indicate that the measured temperature is in degrees Celsius and “F” to indicate that the measured temperature is in degrees Fahrenheit. Additionally, display 28 may include an “M” to indicate that the displayed temperature is from memory. For example, when the thermometer 20 is first turned on, it is desirable to display the last measured temperature in case it was not previously recorded or to remind the user of the prior temperature.

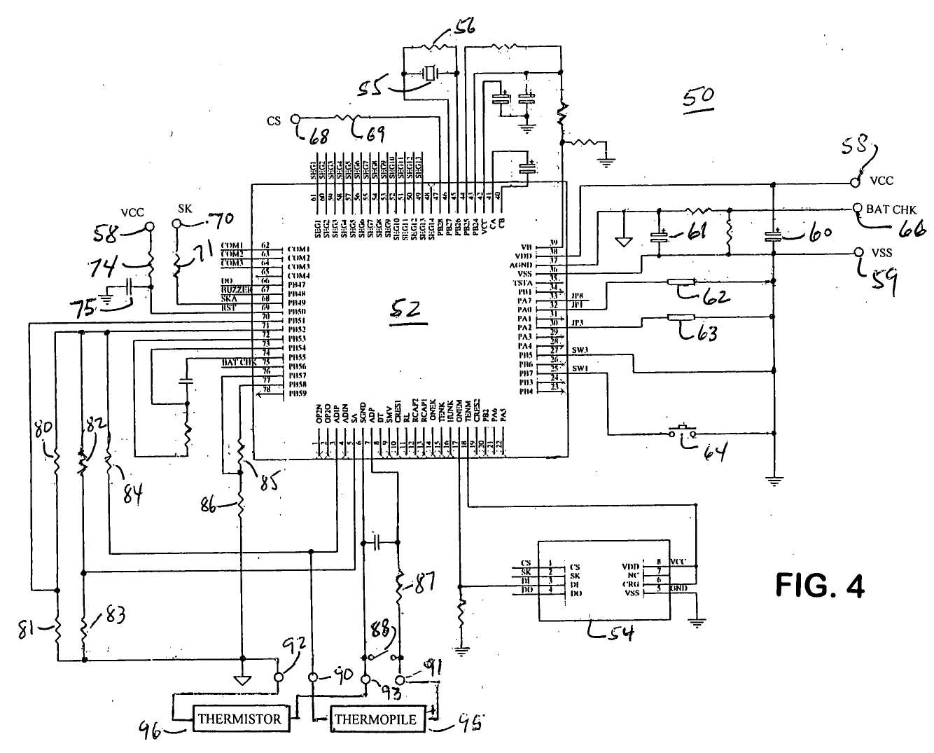

As seen in FIG. 2, the housing 21 may constitute separate portions that are formed from a polymer via well-known thermoplastic molding techniques. In this embodiment, backside 22 may be a unitary housing portion 30 and the front side 23 may comprise an upper housing portion 31 and a lower housing portion 32. Lower housing portion 32 may be removable for access to a battery compartment to periodically replace batteries that supply operating power to internal electronic circuitry 50. The electronic circuitry 50 is further described below in connection with FIG. 4. The housing portions 30-32 engage and secure to each other, such as by interlocking tabs or other known means of snapping thermoplastic housings together. Various other structures are also available for the housing portions 30-32, which would not necessitate a three-part structure for the housing 21.

Preferably, as shown in FIGS. 1-3, housing 21 is tapered near the middle, and is of reduced cross-sectional area thereat, to facilitate better gripping and handling of the thermometer 20. For example, the tapered middle of housing 21 assists in holding thermometer 21 when the user has moist hands by preventing the thermometer from slipping from the grasp of the user. In this respect, the housing 21 may also be provided with a plurality of raised ribs 34, which further assist in retaining the thermometer in the user's hand. In the illustrated embodiment, these ribs are at the lower end of rear housing portion 30, but such ribs may also be disposed along other areas of housing 21, if so desired.

With reference to FIGS. 2 and 3, the upper front housing portion 31 has a tapered or truncated cone-shaped probe 36 projecting outwardly from housing portion 31. For example, housing portion 31 may have a boss 37 integrally molded therewith to which probe 36 attaches. An end or tip 39 of probe 36 is of a size and shape suitable for insertion into the ear of a patient to measure the patient's temperature. Preferably, probe 36 is attached to the front housing portion 31 by a ball and socket connection, and at an oblique angle to the front housing portion 31. Probe 36 can then swivel upward, downward or side to side for ease in inserting the probe into the ear canal of the patient and to permit some movement of the thermometer 20 while the probe 36 is inserted into the patient's ear. An axis 38 may be defined in the longitudinal direction of probe 36. Preferably, probe 36 can also freely rotate about axis 38 for more convenient and comfortable use of the thermometer 20, and to permit rotational movement of the thermometer 20 while the probe 36 is inserted into the patient's ear. The tip 39 of probe 36 has a generally transparent lens or window through which infrared rays pass to temperature sensors in the probe.

In accordance with one aspect of the present invention, illumination is provided at or in front of the tip 39 of the probe 36 to assist in inserting probe 36 into a patient's ear canal to measure the patient's temperature. To this end, an elongated protrusion 40 is formed at or near the top of housing 21, such as at the top ends of housing portions 30 and 31. The location of the light source on the housing 21 is not critical and other locations may also be suitable, such as on the front housing portion 31 below the probe 36, on the sides of front housing portion 31, or the like. However, the top of the housing is preferred because this location generally provides a minimal shadow of the probe tip as the probe 36 is being inserted into the ear canal for thermometers of the type illustrated in FIGS. 1-3 with an obliquely mounted probe.

A light source 42-43 in the form of an incandescent lamp or one or more LEDs are contained in protrusion 40 and are oriented to provide divergent rays 41 of illumination toward and in front of the tip 39 of probe 36. Light source 42-43 thus provides an area of illumination in front of probe 36 to assist in inserting the probe into the patient's ear, especially in lower ambient lighting circumstances, such as in the evening hours or in rooms with low ambient light levels. This also obviates the need to turn on other lighting in the room, which may undesirably disturb the patient.

At least some of the rays 41 of illumination provided by the light source 42-43 will generally intersect with the axis 38 of probe 36 forwardly of the tip 39 of probe 36. If LEDs are used for the light source 42-43, the LEDs are preferably of the bright variety to provide sufficient illumination in the darkest environments. Of course, light source 42-43 is only activated to provide illumination when illumination switch 26 is actuated to the on position, which saves on current drain of the batteries and extends normal battery life.

The electronic circuitry 50 for operating the thermometer 20 is shown in the schematic diagram of FIG. 4. The principal component of circuitry 50 is a microcontroller, generally designated 52, which may be of part number FS9712 commercially available from Fortune Semiconductor Company of Taipei, Taiwan. Of course, other microprocessor or microcontroller part numbers, including from other vendors, may also be suitable for this application. An electrically erasable PROM (EEPROM) memory IC 54 provides non-volatile memory for microcontroller 52. EEPROM 54 is commercially available from Microchip Technology, Inc. of Chandler, Ariz. and other semiconductor manufacturers under industry part number 93LC66. An external crystal 55 and resistor 56 set the clock rate of microcontroller 55 at about 4 MHz.

Positive operating power, such as from a DC battery, is received at terminals VCC 58 for operation of microcontroller 52 and its associated circuitry. Similarly, negative operating power is received at terminal VSS 59. Capacitors 60 and 61 filter the power provided to microcontroller 52. A jumper 62, when closed, sets the circuitry 50 in the calibration mode. When jumper 62 is open, the circuitry 50 is set for the normal temperature measuring mode. A jumper 63, when closed, establishes temperature measurement in the Fahrenheit mode. If jumper 63 is open, temperature measurement will be in the Celsius mode. A switch 64 provides a scan/memory function.

Some of the indicated terminals connect to other terminals in the schematic diagram of FIG. 4. For example, BAT CHK terminal 66 on the right side of the schematic in FIG. 4 connects to the corresponding BAT CHK terminal, pin 75, of microcontroller 52. CS terminal 68 from pin 47 of microcontroller 52, through resistor 69, connects to the corresponding CS terminal, pin 1, of EEPROM 54. Similarly, SK terminal 70, which is connected to pin 68 of microcontroller 52 through resistor 71, is connected to the corresponding SK terminal, pin 2, of EEPROM 54. Upon turning power on, resistor 74 and capacitor 75 provide a slightly delayed reset signal to pin 69 of microcontroller 52 to initialize the microcontroller.

A plurality of resistors 80-87 interface microcontroller 52 with terminals 90-91 that connect to a thermopile 95 and with terminals 92-93 that connect to a temperature sensing thermistor 96. Terminal 91 is connected to the positive terminal of thermopile 95. The thermopile 95 typically measures the temperature of the patient by developing a signal that is proportional to the amount of incident infrared radiation from the ear canal. The thermistor 96 typically measures the ambient temperature. Certain of resistors 80-87 thus couple thermopile 95 to respective terminals of microcontroller 52. Certain of resistors 80-87 similarly couple signals from thermistor 96, which are representative of the measured temperature, back to microcontroller 52 for further analysis of the measured temperature. Switch 88 may be used to check any offset in the temperature measurements, especially during the calibration procedures. It is therefore not accessed by the user during normal operation of thermometer 20.

Certain pins of microcontroller 52 are dedicated to provide information to display 28 and to also provide control signals to display 28. Microcontroller pins 49-61 provide the SEG13-SEG1 signals to display 28, and pins 62-provide 64 the COM1-COM3 signals to display 28. Microcontroller is thus able to supply information and control signals to display 28 to display the measured temperature and the various operating modes and other conditions, including “L” for a low out-of-range temperature, “H” for a high out-of-range temperature and “M” for a temperature previously stored in memory.

It will be understood that the embodiments of the present invention that have been described are illustrative of some of the applications of the principles of the present invention. Various changes and modifications may be made by those skilled in the art without departing from the true spirit and scope of the invention.

1. An ear thermometer for measuring the temperature of a living being by inserting a portion of the thermometer in the ear of the being comprising:
a housing;
a probe attached to the housing, said probe having a tapered tip adapted to be inserted into an ear canal of the being;
a display for displaying a temperature measured by said thermometer; and
a light source disposed on said housing to supply illumination in front of the tip of said probe to illuminate at least the area around the being's ear canal as the tip of the probe is being inserted therein.

2. The ear thermometer in accordance with claim 1 further comprising:
a switch in electrical communication with said light source; and
a source of electrical power,
said switch capable of being actuated to control the application of electrical power to said light source, thereby selectively controlling the light source in suppling illumination.

3. The ear thermometer in accordance with claim 1 further comprising:
a raised protrusion disposed on said housing,
said light source disposed in said raised protrusion and oriented to provide illumination in front of the tip of said probe.

4. The ear thermometer in accordance with claim 3 wherein said raised protrusion is disposed at a top end of the housing of the thermometer.

5. The ear thermometer in accordance with claim 1 wherein the illumination provided by said light source is in the form of divergent rays.

6. The ear thermometer in accordance with claim 1 wherein said light source comprises at least one incandescent lamp.

7. The ear thermometer in accordance with claim 1 wherein said light source comprises at least one light emitting diode.

8. The ear thermometer in accordance with claim 6 wherein said at least one light emitting diode is of the bright light emitting diode type.

9. The ear thermometer in accordance with claim 1 wherein said probe defines an axis and at least some of the illumination from said light source intersects with said axis in front of the tip of said probe.

10. The ear thermometer in accordance with claim 1 wherein the illumination provided by the light source illuminates the entire ear of the being.

11. The ear thermometer in accordance with claim 1 wherein said probe is pivotally mounted to said housing.

12. The ear thermometer in accordance with claim 1 wherein said probe is rotationally mounted to said housing.

13. The ear thermometer in accordance with claim 1 wherein said probe is mounted to said housing at an oblique angle thereto.
